(19) United States
(12) Patent Application Publication
(10) Pub. No.: US 2010/0234463 A1
(43) Pub. Date: Sep. 16, 2010

(54) Method for Identifying Modulators of Gamma Secretase or Notch

(76) Inventors: Ian Churcher, Herts (GB); Peter Brian Simpson, Cheshire (GB); Carmel Beulin Nanthakumar, Middlesex (GB)

Correspondence Address: MERCK, P O BOX 2000, RAHWAY, NJ, 07065-0907, US

(21) Appl. No.: 11/991,586

(22) PCT Filed: Sep. 1, 2006
(86) PCT No.: PCT/GB2006/050269

§ 371 Date: Mar. 6, 2008

(30) Foreign Application Priority Data
Sep. 7, 2005 (GB) 0518232.4

(51) Int. Cl. A61K 31/192; C40B 30/06; A61P 25/28; A61P 35/00

(52) U.S. Cl. 514/570; 506/10

(57) ABSTRACT

The present invention relates to methods for high throughput screening to identify selective modulators of γ-secretase. Such modulators selectively block γ-secretase activity without affecting Notch cleavage and signaling. Said methods employ the use of high content screening methodologies to measure and quantify the translocation of the Notch intracellular domain (NICD) between the nucleus and cytoplasm in cells and the activation of Notch.

The present invention relates to methods for high throughput screening to simultaneously identify modulators of γ-secretase and Notch and their use in the treatment of conditions in which abnormal activity of γ-secretase or Notch is implicated including Alzheimer's disease, multiple sclerosis and cancer.

Extracellular deposition of amyloid-β (Aβ) is a defining feature of Alzheimer's disease (AD), a neurodegenerative disorder of the central nervous system. Aβ accumulates in the brain resulting in plaque formation and impaired brain function. Specifically, Aβ₄₀ and Aβ₄₂ cleavage products generated by proteolytic processing of the β-amyloid precursor protein (βAPP) are the main constituents of senile plaques found in individuals with AD.

The aspartyl protease, γ-secretase is one of two enzymes responsible for the sequential processing of βAPP. An inhibitor of γ-secretase may reduce levels of Aβ₄₀ and Aβ₄₂ and therefore delay or prevent the progression of AD. However, a potential mechanism-based side effect of catalytic site-directed γ-secretase inhibition is impaired Notch signaling.

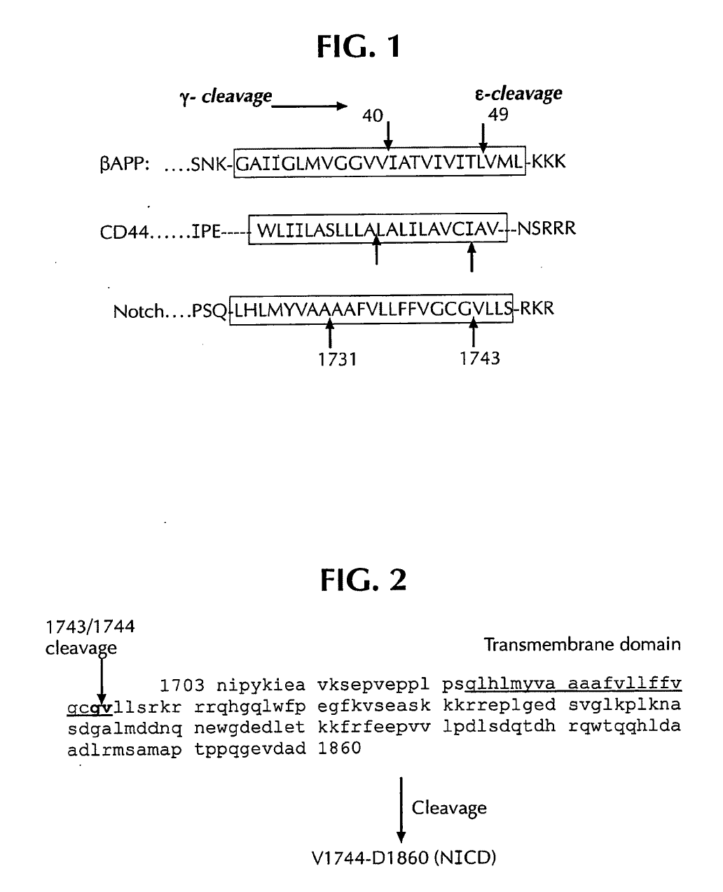

Notch exists as a membrane-bound receptor and is activated following binding of the Delta/Serrate/Jagged family of ligands, FIG. 1. The Notch intracellular domain (NICD) is released following a cleavage event at site 3 (S3) thought to be mediated by the γ-secretase complex, De Strooper, B., et al., Nature 398: 518-522 (1999). Following S3 cleavage, NICD translocates to the nucleus and binds directly to downstream transcription factors to elicit biological effects, Fortini, M., Nature Reviews 3: 673-684 (2002).

More recently, a gain of function mutation resulting in over-activation of the Notch pathway has been implicated in T cell acute lymphoblastic leukemia, Weng, A. P. et al., Science 306: 269-271 (2004). As such, compounds which modulate Notch signaling may be beneficial for the treatment of cancers where aberrant Notch signaling is implicated. Notch is also a potential therapeutic target for the treatment of multiple sclerosis (MS) and has been implicated in the limited remyelination in MS, John, R. J. et al., Nature Medicine 8: 1115-1121 (2002).

The Notch nuclear translocation assay herein uses a cell line that over-expresses NotchΔE, a truncated version of Notch which undergoes constitutive S3 cleavage in the absence of ligand stimulation, Schroeter, E., et al., Nature 393: 382-393 (1998). Translocation assays, such as the one described herein, detect movement of the protein of interest, i.e. NICD, from the cytoplasm to the nucleus and can be used to identify compounds which block this transition.

Thus, it is an object of this invention to identify therapeutic agents that selectively block γ-secretase activity without affecting Notch cleavage or, vice versa, affect Notch activity while exhibiting no effect on γ-secretase. The present invention relates to a high throughput screening assay that was developed to rapidly identify such selective γ-secretase and Notch modulators.

In one embodiment, the present invention is a method for identifying selective γ-secretase modulators and Notch inhibitors using high content screening analysis of the translocation of a Notch intracellular domain (NICD) between the nucleus and cytoplasm in cells, which screening comprises:
(a) providing an array which contains multiple cells that express NICD and that are labeled with a detectable epitope tag;
(b) contacting the cells in said array with at least one test compound;
(c) imaging said cells in each location of the array with fluorescence optics;
(d) converting the optical information into digital data;
(e) analyzing said digital data to determine the degree of fluorescence in the nucleus and cytoplasm of said cells;
(f) calculating the test compound-induced changes to indicate one of the following:
   i. a difference between the fluorescence from the reporter molecules in the cytoplasm from the fluorescence from the reporter molecules in the nucleus;
   ii. a ratio of fluorescence from the reporter molecules in the cytoplasm to the fluorescence from the reporter molecules in the nucleus; and
      wherein test compound-induced changes indicate an effect of the test compound on the translocation of NICD between the cytoplasm and the nucleus in the cells.

In one embodiment of the invention such modulators are identified using a high content screening assay to simultaneously measure Aβ₄₀, Aβ₄₂ and Notch. In another embodiment of the invention a test compound that exhibits an IC₅₀ value for inhibition of Aβ₄₂ and/or Aβ₄₀ while exhibiting no effect on Notch nuclear translocation is identified as a selective γ-secretase inhibitor. In still another embodiment of the invention a test compound that exhibits an IC₅₀ value solely for Notch inhibition and exhibiting no effect on Aβ₄₀ and Aβ42 is identified as a selective Notch inhibitor.

A further embodiment of the invention is a method for identifying selective Notch modulators using high content screening analysis of the activation of Notch, which screening comprises:
(a) providing an array which contains multiple cells that express full length Notch or cells capable of undergoing S2 ligand-dependent cleavage and that are labeled with a detectable fluorescent reporter molecule;
(b) pre-incubating said cells with at least one test compound;
(c) contacting said cells with at least one Notch binding ligand selected from the group consisting of Delta, Serrate and Jagged;
(d) imaging said cells in each location of the array with fluorescence optics;
(e) converting the optical information into digital data;
(f) analyzing said digital data to determine the degree of fluorescence in the nucleus and cytoplasm of said cells;
(g) calculating the test compound-induced changes to determine one of the following:
   i. the difference between the fluorescence from the reporter molecules in the cytoplasm from the fluorescence from the reporter molecules in the nucleus;
   ii. the ratio of fluorescence from the reporter molecules in the cytoplasm to the fluorescence from the reporter molecules in the nucleus; and
      wherein test compound-induced changes identified in step (g) indicates i) an increase in the level of nuclear translocation of Notch in the cells pre-incubated with the test compounds as compared to control cells such that the test compound is a Notch receptor agonist, or ii) a decrease in the level of nuclear translocation of Notch in the cells pre-incubated with the test compounds as compared to control cells such that the test compound is a Notch receptor antagonist.

The selective γ-secretase modulators identified herein may be used for the treatment of Alzheimer's disease by administering to a patient in need of said treatment thereof an effective amount of said selective γ-secretase modulator. Similarly, selective Notch inhibitors identified herein may be used for the treatment of multiple sclerosis and cancer by administering to a patient in need of said treatment thereof an effective amount of said selective Notch inhibitor.

FIG. 1 is a schematic diagram illustrating the proteolytic regulation of the Notch receptor which results in the generation of the Notch intracellular domain (NICD). Notch undergoes three cleavage events: the precursor is cleaved at a luminal site by a furin-like convertase to generate extracellular and intracellular/transmembrane domains; the domains are rejoined to form a heterodimeric form of Notch; binding of the Delta/Serrate/Jagged ligands results in ectodomain removal, which is membrane anchored and subsequently processed by γ-secretase.

FIG. 2 depicts images of cells captured using the Cellomics™ ArrayScan® (Cellomics Inc, Pittsburgh, Pa.) demonstrating cytoplasmic subcellular localization of NICD following treatment with a γ-secretase inhibitor. Cells seeded at 25,000 cells/well were treated with 1% DMSO (FIG. 2A-2D) or 1% DMSO containing 300 nM compound A for three hours (FIG. 2E-2H). Fixed cells were stained for c-myc-tagged NICD (FIGS. 2B and 2F) and counterstained with Hoechst 33342 (FIGS. 2C and 2G) for visualization of nuclei. Images were acquired using the ArrayScan® 4.0 and ×20 objective. The Hoechst 33342 staining was used to identify objects and a blue nuclear mask applied to accepted nuclei (FIGS. 2A and 2E). Alexa 488 and Hoechst 33342 merged images (FIGS. 2C and 2G) were enlarged further (FIG. 2D and FIG. 2H).

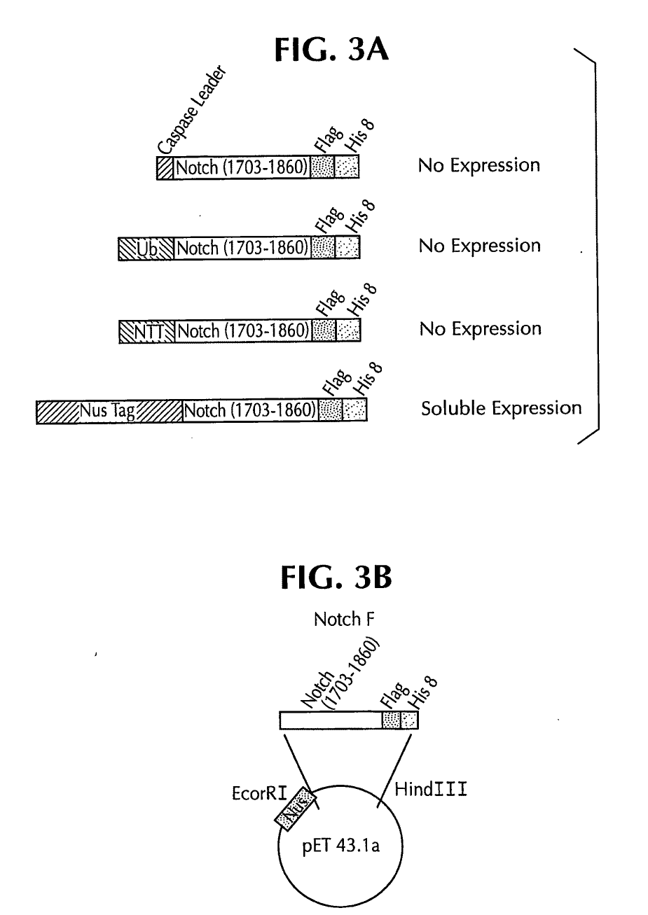

FIG. 3 shows the kinetics and dose dependant inhibition of NICD nuclear translocation by a γ-secretase inhibitor. HEK293 cells stably expressing NotchΔE were treated with increasing concentrations of Compound B up to 100 nM at various times up to three hours. Cells were fixed and immunostained for NICD using an antibody against c-myc. NICD immunoreactivity in the cytoplasm and nucleus was quantitated using the ArrayScan® 4.0. Each point represents the mean±SEM for four replicates.

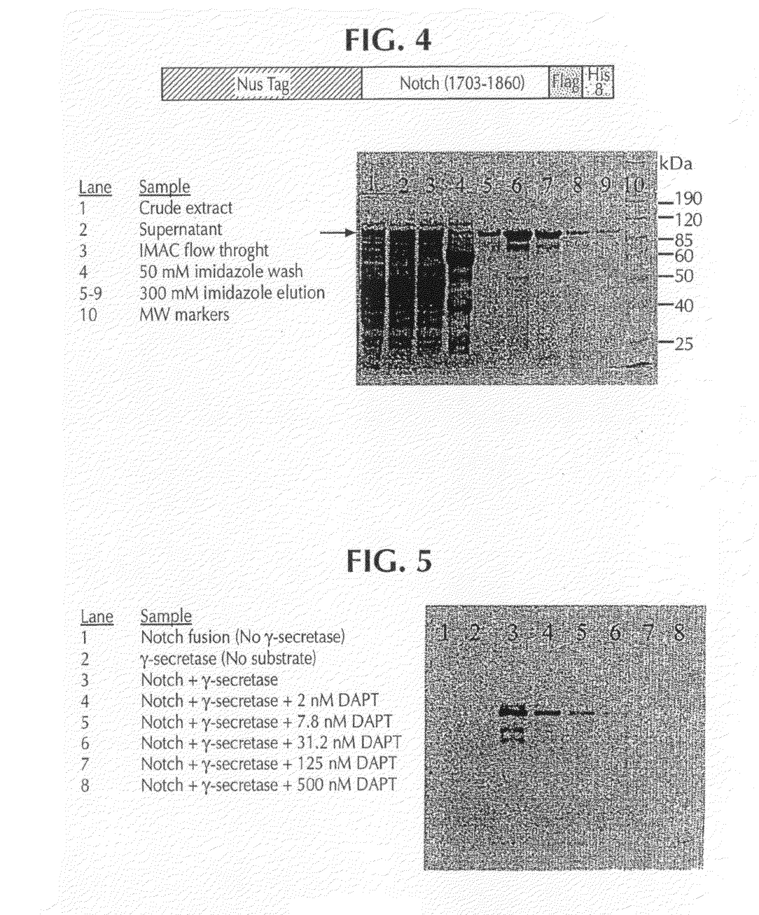

FIG. 4 shows the subcellular localization of NICD over time following treatment with compound C. HEK293 cells expressing NotchΔE were treated with 10 μM compound C for up to twenty four hours prior to fixing and immunostaining with a monoclonal antibody to detect c-myc-tagged NICD. Each bar represents the mean±SEM for eight replicate values.

FIG. 5 shows the parallel detection of Aβ₄₀, Aβ₄₂ and NICD nuclear translocation in the same cell population. HEK293 cells co-expressing βAPP₆₉₅ and NotchΔE were treated with compound D up to 1 μM for twenty four hours. Cell-conditioned media was removed and Aβ peptides quantified by ECL. The cell monolayer was fixed and immunostained with a monoclonal antibody to detect c-myc-tagged NICD. Each point represents the mean±SEM for four replicate values.

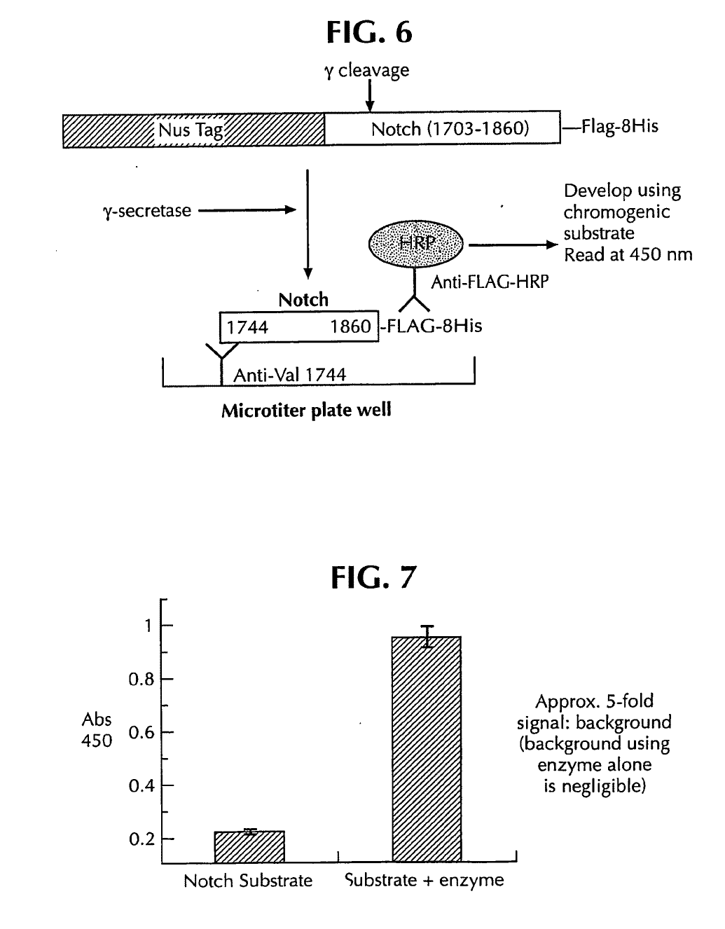

FIG. 6 demonstrates that NSAIDs selectively inhibit Aβ₄₂ secretion without affecting Notch nuclear translocation. HEK293 cells stably expressing βAPP₆₉₅ and NotchΔE were incubated with increasing concentrations of NSAID, compounds E and F, (FIGS. 6A and 6B, respectively) or a γ-secretase inhibitor, compound G (FIG. 6C), for twenty four hours. Levels of Aβ peptides in cell-conditioned media were quantified by ECL and cells immunostained for detection of NICD using anti-c-myc antibody clone 9E10. Data is expressed as a percentage±SEM of vehicle control values for 4 replicates. IC₅₀ values for inhibition of Aβ₄₀, Aβ₄₂ and NICD translocation were calculated by nonlinear regression fit analysis using GraphPad Prism software.

FIG. 7 shows the identification of novel compounds exhibiting selective dual inhibition of Aβ₄₀ and Aβ₄₂. HEK293 cells stably expressing βAPP₆₉₅ and NotchΔE were incubated with increasing concentrations of compound (FIG. 7A) or compound I (FIG. 7B) for twenty four hours. Levels of Aβ peptides in cell-conditioned media were quantified by ECL and cells immunostained for detection of NICD using an anti-c-myc antibody, 9E10. Data is expressed as a mean percentage±SD of four replicates when compared with vehicle control values. IC₅₀ values for inhibition of Aβ₄₀, AB₄₂ and NICD translocation were calculated by nonlinear regression fit analysis using GraphPad Prism software (Table 1).

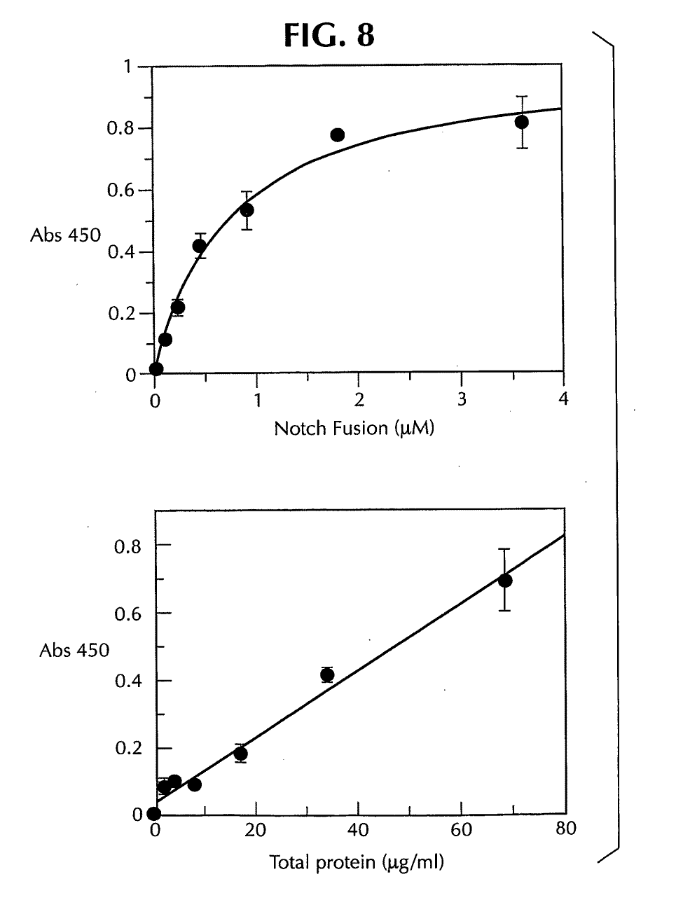

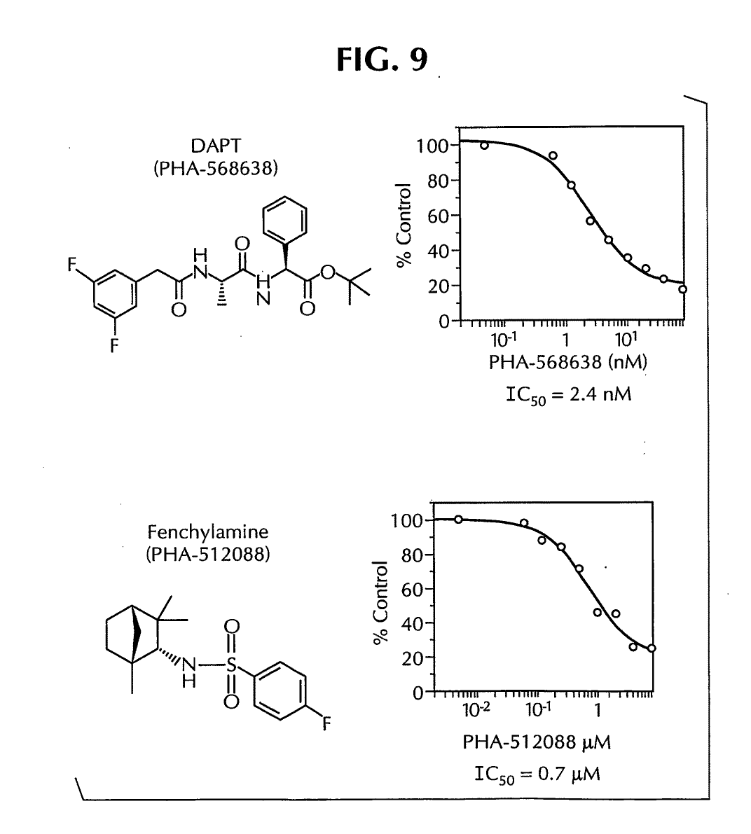

FIG. 8 is a schematic for the use of the translocation assay of the present invention to identify selective modulators and to infer the compound mechanism of action. By comparing the IC₅₀ values obtained for Aβ₄₀, Aβ₄₂ and Notch, compounds can be classified and prioritised for further work based on the selectivity profiles obtained.

The term “high content screen” in the context herein refers to an assay that correlates the temporal and spatial distribution of a fluorescently labeled protein of interest (POI) within a cell with the structure and function of said POI by measuring a subcellular event. In a specific embodiment of the instant invention, the subcellular event is a translocation, i.e. the movement of the POI from the cytoplasm to the nucleus of a cell. Such high content screens generally utilize an automated image capture methodology and the application of software algorithms to detect the subcellular events. A “high content screening system” refers to an integrated platform system, such as Cellomics™ ArrayScan® (Cellomic Inc, Pittsburgh, Pa.), that uses a computerized cytometric system to analyze the translocation of a POI in multiple cells in microtitre plate wells simultaneously with image acquisition.

The term “modulator” refers to therapeutic agents that have an effect on the generation of Aβ peptides or the Notch receptor signaling pathway. Such modulators may act to activate, i.e. up-regulate, as well as to inhibit, i.e. down-regulate said functions.

The term “therapeutic agent” refers to any compound including, but not limited to, small molecules, peptides, nucleic acids or other biologics, that can be administered to treat the diseases herein. The therapeutic agent may be included within a formulation or pharmaceutical composition.

The term “selective γ-secretase modulator” refers to a therapeutic agent which inhibits Aβ₄₂ secretion while exhibiting no effect on Notch mediated signaling.

The term “selective APP modulator” refers to a therapeutic agent which inhibits APP proteolysis while exhibiting no effect on γ-secretase activity.

The term “selective Notch inhibitor” refers to a therapeutic agent which inhibits Notch activity while exhibiting no effect on Aβ₄₀ and Aβ₄₂ secretion.

The term “allosteric inhibition” describes those compounds binding to a novel enzyme site which is discrete from the catalytic binding site.

The process of signal transduction is generally complex and typically involves the activation and translocation of macromolecules from the cytoplasm to the nucleus. These macromolecules may be transcription factors, such as NF-κB, NFAT, C-jun and STATs, that are activated and processed to the nucleus. Accordingly, the measurement of transcription factor movement from the cytoplasm to the nucleus can be used as a direct measure of transcription factor activity. High content screening technology permits the identification of protein localization in subcellular compartments, such as the cytoplasm and the nucleus, as originally demonstrated by colleagues of the Applicants (Ding, G., et al, J. Biol. Chem. 273(44): 28897-905, 1998) for the NF-κB nuclear translocation induced by interleukin-1 and tumor necrosis factor-α. Applicants herein have now utilized high content screening methodology to localize an intracellular fragment of Notch (NICD). Levels of the NICD within the cytoplasm of a cell line developed in-house were used as an index of Notch inhibition, a common effect of γ-secretase inhibitors targeted for Alzheimer's disease (AD). The effects ensuing from such Notch inhibition on the pathway in vivo may lead to a variety of undesirable side-effects, including effects on bone marrow and development. There is therefore a need for in vitro assays which characterize the ability of medicinal chemistry lead compounds to exhibit a propensity for Notch inhibition in vivo, in order to identify therapeutic agents which are potential selective inhibitors of the activity of γ-secretase, but not Notch. The assay of the instant invention is used for the identification of such selective inhibitors of Notch by quantifying levels of the Notch intracellular fragment (NICD) within the nuclear or cytosolic compartments using an antibody against a c-myc label appended to Notch during the development of the cell line. The instant assay is ligand independent as the cells undergo constitutive S3 cleavage to generate the NICD fragment and levels of Aβ40 and Aβ42 are also measured simultaneously in the same cell sample.

Conventional assays to measure Notch cleavage rely on labor-intensive, low throughput methods, for example, Western blots, that require extensive human interaction. Creating data or information from which determinations can be made as to biological mechanism results from many small scale experiments often performed over many weeks or months. As such, these techniques cannot be efficiently utilized for routine or rapid assays. In contrast, high content screening generally requires much fewer cells, is much more rapid, and generates automatically quantified datasets, enabling high numbers of compounds to be simultaneously evaluated for activity against Aβ₄₀, Aβ₄₂ and Notch.

In the instant invention, through the use of a high content screening methodology, the degree of immunofluorescence in cytosolic and nuclear compartments can be automatically measured and their ratio calculated, such that compounds affecting this ratio can be quantified for potency. Such a high content screening assay can be performed, for example, on the Cellomics™ ArrayScan® (Cellomics, Pittsburgh, Pa.) using HEK293 cells that overexpress βAPP and NotchΔE. Inhibition of NICD translocation is directly detected by the measurement of increased levels of c-myc-tagged NICD immunoreactivity in the cytosolic compartment. Similar measurements can be made using an IN Cell Analyzer 1000 and 3000 (Amersham Biosciences, Piscataway, N.J.), Pathway HT™ (Atto Bioscience, Rockville, Md.), ImageXpress® 5000A (Molecular Devices Corporation, Union City, Calif.), iCyte™ Automated Imaging Cytometer (CompuCyte, Cambridge, Mass.), EIDAQ™ 100-HTM (Q3DM, San Diego, Calif.), Discovery-1™ High Content Screening System (Universal Imaging Corporation/Molecular Devices, Downingtown, Pa.), Abraham, V., et al., TRENDS in Biotech., 22 (1): 15-22 (January 2004), Acumen Explorer (TTP LabTech Ltd, Royston, Herts)

High content screening systems, such as the ArrayScan® identified above, acquire images of cells from multiple fields from well to well of a microtitre plate and applies a user-optimized software algorithm to generate quantitative data. The cytoplasm to nuclear translocation algorithm identifies cell nuclei using a detectable epitope tag, such as a fluorescent DNA stain, for example, Hoechst 33342, imaged in a different wavelength from the protein of interest. This fluorescence is used to define the nuclear compartment of the cell. The algorithm applies a mask to this region and determines the fluorescence intensity of the protein of interest in the nucleus versus the cytoplasm. The cytoplasm is defined by the area within a pair of concentric rings immediately outside the nuclear mask. A dilated ring beyond this compartment, of dimensions predefined by the user, can measure cellular fluorescence from a sample region of the cytoplasm. The fluorescence intensity in the cytoplasm is subtracted from that in the nucleus to give a translocation score, see, for example, Ding, G., et aL, J. Biol. Chem. 273 (44):28897-28905 (1998). Alternatively, the total staining in the sample area of the cytoplasm can be used to generate a translocation score, for example, if levels of the protein of interest are low in the cytosol, this would suggest the protein has translocated to the nucleus, and vice versa. Assays of this type have been validated with a large number of in-house medicinal chemistry compounds by comparing IC₅₀ values obtained using a western blot method to measure NICD with the nuclear translocation IC₅₀ values and a rank order of potency for a set of inhibitors obtained.

Potential applications of the high content screening assay of this invention include, but are not limited to, the identification of selective Notch modulators for therapeutic agents to treat multiple sclerosis and cancer and the assessment and quantification of Notch inhibition by γ-secretase inhibitors for therapeutic agents to treat Alzheimer's disease.

To identify therapeutic agents that promote Notch activation, cells expressing full length Notch, or cells capable of undergoing S2 ligand-dependent cleavage, are required. In some embodiments of the invention several c-myc tags or the like may be attached to the POI to facilitate detection of the NICD through the use of known antibodies to c-myc (clone 9E10, Oncogene Research Products, San Diego, Calif., clone 4A6, Upstate, Lake Placid, N.Y. and clone 9E10, Santa Cruz Biotechnology Inc, Santa Cruz, Calif.) or untagged NICD (activated Notch-1, Abcam Ltd, Cambridge, Cambridgeshire, cleaved Notch-1, Cell Signalling Technology, Beverley, Mass.). Potential receptor agonists are pre-incubated with cells expressing the POI and compared with control cells exposed to media alone. Upon exposure to Notch activators an increase in the level of nuclear translocation of NICD compared with translocation observed in the control cells would suggest activation of the Notch pathway. As a positive control, the Delta/Serrate or Jagged family of ligands could be used to stimulate Notch signaling. This latter approach could also be used for Notch antagonist screening, by pre-incubating cells with test compounds and then activating the Notch pathway by adding either Delta/Serrate or Jagged and quantifying ligand-induced nuclear-translocation of Notch.

In addition to cells that undergo constitutive S3 cleavage to generate a NICD fragment, the cell line used herein by Applicants over-expresses the β-amyloid precursor protein (βAPP) in order to be able to simultaneously detect amyloid peptides, Aβ40 and Aβ42. The HEK293 cell line overexpressing βAPP₆₉₅ was generated using the methodology of Shearman, M., et al., Biochemistry 39:8698-8704 (2000) and the NotchΔE construct was subsequently transfected into this cell line as described by Beher, D., et al., J. Biol. Chem. 276 (48): 45394-45402 (2001). The βAPP₆₉₅ construct represent human, full-length APP₆₉₅. Those skilled in the art would know how to select and use other constructs containing the γ-secretase cleavage site as an alternative substrate, such as APP₇₇₀.

In some embodiments of the invention the cell conditions and the existing translocation algorithm was modified to permit the imaging of HEK293 cells. In order to simultaneously measure Notch Aβ₄₀ and Aβ₄₂, a cell line expressing both APP and Notch and that also expresses endogenous γ-secretase was utilized. An HEK293 βAPP695NotchΔE cell line was developed as described above. Generally, HEK293 cells are difficult to image as they do not grow as single isolated cells, but over time cluster together and form groups, which interferes with image analysis in this type of screening. In the assay herein, the cell conditions were modified: 1) by improving cell adherence to the plate for the duration of the assay by plating the cells on poly D-lysine coated plates; 2) by optimizing cell density by spatially separating the cells for imaging; and 3) by reducing the serum concentration to 0.4% to minimize cell proliferation and keep cells quiescent.

The translocation algorithm used herein was modified by applying a threshold approach based on nuclear size and shape of the cells and the level of NICD fluorescence intensity. The first threshold required the identification and selection of a suitable pre-defined algorithm analogous to the biological activity to be measured, for example, a nuclear to cytoplasm translocation. The pre-defined algorithm was then modified to ensure that the correct cells and changes were detected. To modify the pre-defined algorithm, the nuclei to include in the analysis were defined. As described above, nuclear size and shape were optimized to ensure only single cells were analyzed; if the nuclear size was too small or irregular, those cells were rejected from the analysis. Providing that the cells satisfied the criteria for nuclear size and shape, a second threshold was applied based on NICD fluorescence intensity. Notwithstanding that an engineered cell line was employed for this assay and that the cells were grown under antibiotic selection, some cells did not express NICD. Cells that did not express NICD were identified as they stained with the nuclear marker, Hoechst 33342, but did not fluoresce in the second, NICD channel. Cells not expressing NICD were excluded from the analysis by setting a value for NICD fluorescence and only those cells exhibiting a fluorescence intensity at and above that value were analyzed. An exemplary minimum fluorescence intensity value is 250.

The instruments generally used for high content screens are set by the user to count at least 100 single cells to achieve statistical power. In addition to the above listed conditions, by plating the HEK293 cells on the day of the assay and incubating at 37° C. for a maximum of three hours, the cells were given the minimal time required to adhere to the plate prior to compound addition. Those skilled in the art using standard molecular biology techniques would know how to select and use cell types other that HEK293 that express endogenous γ-secretase for the assay herein, including, but not limited to transfected chinese ovary hamster (CHO), SH-SY5Y and HeLa or other cell lines expressing detectable levels of endogenous βAPP and Notch.

### EXAMPLES

1. Detection Antibodies

Two antibodies from Cell Signaling Technology and Abcam Ltd (product codes 2421S and ab8925 respectively) recognizing the N-terminal sequence of the cleaved Notch intracellular domain (NICD) were initially evaluated for use, but failed to detect nuclear translocation using standard immunocytochemical methods (Cell Signaling Technology, Inc., Beverly, Mass. 1:100, 1:500, 1:1000; Abcam® Ltd., Cambridge UK, 1:100, 1:500, 1:1000). A monoclonal antibody against c-myc, clone 9E10 (Oncogene Research Products, Calbiochem®, Merck Bioscience, Ltd., Nottingham, UK, 1:125) was successfully used to detect the c-myc-tagged NICD. For fluorescence detection, goat anti-mouse IgG conjugated to Alexa-488 was used in combination with Hoechst 33342 (Invitrogen™, Carlsbad, Calif., 1:500 and 1 μg/m1 respectively). For quantification of amyloid peptides, a biotinylated monoclonal antibody specific for amino acid residues 17-24 of human Aβ peptide (Clone 4G8, Signet Laboratories, Dedham, Mass., 1:250) was used in combination with monoclonal antibodies recognizing either the C-terminal fragments of Aβ₄₀ or Aβ₄₂ (Clarke, E. E. and Shearman, M. S., J. Neuros Meths. 102: 61-68 (2000)).

2. Cell Cultures

Human embryonic kidney (HEK) 293 cells stably over-expressing β-amyloid precursor protein (βAPP₆₉₅) and c-myc-tagged NotchΔE (M1727V) have been previously described (Beher, D. et al., J. Biol Chem. 276: 45394-45402, (2001)). Cells were routinely maintained in Dulbecco's modified Eagle's medium (DMEM) supplemented with 10% (v/v) fetal calf serum (FCS, Invitrogen™, Carlsbad, Calif.), 1 μg/ml puromycin (Sigma-Aldrich, St. Louis, Mo.) to maintain βAPP expression and 100 μg/ml zeocin (Life Technologies, Inc., Rockville, Md.) to select for NotchΔE expression. Cells were passaged on a weekly basis using 1:10 split ratio.

On the day of the assay, media was removed and cells washed with phosphate-buffered saline (PBS, Sigma-Aldrich, St. Louis, Mo.), followed by incubation with 1.5 ml Accutase (PAA Laboratories GmbH, Pasching, Austria) at 37° C. for 10 minutes to detach cells. DMEM supplemented with 10% (v/v) FCS was added and the cell suspension centrifuged for 5 minutes at 1120 prm. The supernatant was discarded and cell pellet re-suspended in 1 ml DMEM containing 0.4% (v/v) FCS. A single cell suspension was obtained by passing the suspension through a 1 ml pipette tip ten times. A further 9 ml DMEM supplemented with 0.4% (v/v) FCS was added and cells counted.

3. Translocation Experiments

Cells were plated in poly-d-lysine coated 96-well plates (Becton Dickinson, Fullerton, Calif.) at 10,000 cells/well in DMEM supplemented with 0.4% (v/v) FCS (Invitrogen™, Carlsbad, Calif.). After three hours incubation at 37° C., experiments were performed by incubating cells for 24 hours at 37° C. with inhibitors in the presence of 1% v/v DMSO final. For dose response experiments, compounds were serially diluted in DMSO to 100× final concentration range, followed by parallel dilution in serum-free DMEM to give 10× final concentration range. Compounds were further diluted 1 in 10 upon addition to cell plates. For time course experiments, cells were incubated with test compounds at various time points from zero to 24 hours at 37° C. For control wells either vehicle (DMEM containing 1% v/v DMSO) or the specific γ-secretase inhibitor, L-685, 458 (Shearman, M. S. et al., Biochem. 39: 698-8704 (2000)) was added to cells to give 10 μM final concentration. Cell conditioned media was removed for quantification of Aβ peptides and the cell monolayer fixed by incubating with 3.7% (v/v) formaldehyde (Sigma-Aldrich, St. Louis, Mo.) in PBS at room temperature for fifteen minutes.

4. Indirect Immunofluorescence

Cells were washed with PBS (three times, five minutes each) and incubated with 0.1% Triton-X in PBS for 90 seconds at room temperature. After washing with PBS (three times, five minutes each), fixed cells were incubated with the primary antibody diluted in PBS for one hour at room temperature. After washing with PBS (three times, five minutes each), the cells were incubated with fluorescently labelled secondary antibodies and Hoechst 33342 diluted in PBS, for one hour at room temperature. After washing with PBS (three times, five minutes each), the plates were sealed and transferred to a Cellomics™ ArrayScan® 4.0 (Cellomics Inc, Pittsburgh, Pa.) for quantification of NICD translocation.

5. NICD Data Acquisition

Automated image capture and quantification of NICD nuclear translocation was performed using a Cellomics™ ArrayScan® 4.0 and the Cytoplasm to Nucleus Translocation BioApplication software (Cellomics, Pittsburgh, Pa.). This BioApplication software enables the measurement of fluorescence intensity within the nucleus and a pre-defined area of the cell cytoplasm. The algorithm was modified to scan 100 cells/well using Hoechst 33342-stained nuclei for object identification. A threshold value was applied to the fluorescence intensity values obtained for the NICD to ensure that image analysis was only performed on cells expressing the NotchΔE construct. Data was analyzed using values obtained for either 1) the MeanCytoRinglnten (TargetCh) parameter, which is a measure of the total fluorescence intensity within a sample area of the cell cytoplasm, or 2) the MeanNuc-CytolntenDiff, which subtracts the values for fluorescence intensity in the cytoplasm from total nuclear fluorescence. The number of cells counted was determined from values obtained for the “ValidObjectCount” and the “# Valid” parameters. Values for % inhibition were calculated by measuring NICD fluorescence intensity in cells exposed to vehicle or to a test compound, for example, compound C, for determination of maximal and minimal NICD nuclear translocation, respectively.

6. Quantification of Aβ Peptides in Conditioned Cell Culture Media

Aβ peptides secreted into the media were quantified using an electrochemiluminescence (ECL) assay in a 96-well plate format (Sector HTS™ analyzer, Meso Scale Discovery® a division of Meso Scale Diagnostics™, LLC (MSD™), Gaithersburg, Md.). Cell supernatants (50 μl each for Aβ₄₀ and Aβ₄₂) were transferred to a coated Multi-Array™ 96-well plates (Meso Scale Discovery) and agitated for 24 hours at 4° C. with antibody pairs to detect Aβ₄₀ and Aβ₄₂ in PBS containing 2% (w/v) Bovine Serum Albumin (BSA, Sigma-Aldrich, St. Louis, Mo.) and 0.2% (v/v) Tween-20 (Sigma-Aldrich, St. Louis, Mo.). Plates were washed with PBS (three times, five minutes each) and media replaced with 150 μl Read buffer S (Meso Scale Discovery®) prior to detecting Aβ₄₀ and Aβ₄₂ using the Sector HTS™ Analyzer. Non-specific background values were determined from the signal obtained when cells were exposed to 10 μM compound C and compared with levels of Aβ₄₀ and Aβ₄₂ in cells incubated with the vehicle control (1% DMSO v/v).

7. Data Analysis

Mean values for vehicle control and compound C treated cells were calculated. The standard error of mean (SEM) and Coefficient of Variance (CV) was also determined to ensure the spread of data values was within an acceptable range. Percent control values were calculated using the following formula:

((X-Mean compound Y)/(Mean Vehicle))*100

where:

X is the experimental value;

Mean compound Y is the mean value obtained for cells exposed to compound Y; and

Mean Vehicle is the mean value calculated for cells exposed to vehicle control.

For dose response experiments, percent control values were expressed as mean±SEM for a minimum of four replicates and plotted against the logarithm of the test compound concentration. Non-linear regression analysis using a four-paramater fit model was performed using GraphPad Prism (GraphPad Software, Inc., San Diego, Calif.) to obtain the median inhibitory concentration (IC₅₀), i.e., the concentration of inhibitor required to reduce the specified response by 50%.

8. Nuclear Translocation of NICD

The nuclear translocation of NICD was investigated using HEK293 cells over-expressing a c-myc-tagged, truncated Notch construct undergoing constitutive S3 cleavage in the absence of ligand stimulation. Cells were incubated with γ-secretase inhibitors (compounds A and B) for up to twenty four hours and the subcellular localization of NICD detected using immunofluorescence. Images were captured and quantified using the ArrayScan® 4.0. In cells exposed to vehicle alone, NICD immunoreactivity was predominantly nuclear and co-localized with Hoechst 33342 (FIGS. 2B and 2C), suggesting NICD translocation to the nucleus under basal conditions as expected. There was no difference in Hoechst 33342 nuclear staining between vehicle and compound treated cells (FIGS. 2A and 2E). Catalytic site-directed γ-secretase inhibitors have been reported to inhibit Notch S3 cleavage (Lewis, H. D., et al., Biochem 42: 7580-7586 (2003)) and therefore nuclear translocation of NICD should be compromised in the presence of such inhibitors. To test this, compounds (compounds A and B) demonstrating γ-secretase inhibition were selected from an in-house compound collection and profiled for effects on Notch nuclear translocation. In cells treated with compound A for three hours, NICD was detected in the cytoplasm only and did not co-localize with Hoechst 33342 (FIGS. 2F and 2G), consistent with compound-mediated inhibition of NICD nuclear translocation. Significant immunofluorescence was not detected when the primary antibody to c-myc was omitted from the staining procedure (data not shown). NotchΔE non-expressing cells were identified by Hoechst-stained nuclei and the lack of NICD immunostaining in either the nucleus or cytoplasm (FIGS. 2C and 2G).

NICD staining patterns in the absence and presence of compound were quantitated using the Cytoplasm to Nucleus BioApplication software (Cellomics Inc, Pittsburgh, Pa.). Initially, cells were identified using the Hoechst 33342 stain and the object selection parameters modified to image HEK293 cells. Over time, HEK293 cells typically associate into clusters of cells rather than remaining spatially separated and this presents significant problems for image analysis. To overcome this, a maximum nuclear size threshold was applied such that a nuclear area greater than 437 pixels was excluded from the analysis. This was combined with a reduced serum concentration and low cell seeding density to minimize cell aggregation. A second fluorescence intensity threshold was applied for NICD immunofluorescence so that non-expressing cells were discounted from the analysis. A fluorescence intensity value of 250 was set as the minimum required value denoting significant NICD immunoreactivity.

Cells were treated with increasing concentrations of a second γ-secretase inhibitor, compound B for various time points up to three hours (FIG. 3). After thirty minutes, a trend for dose-dependent inhibition of NICD nuclear translocation was apparent, ranging from 147±10 at the lowest concentration of compound B to 78±10 with 100 nM of compound B. Maximal inhibition of NICD nuclear translocation occurred after 120 minutes, ranging from 123±2 to -1±3 at the lowest and highest concentrations of compound B, respectively. Gamma-secretase, an aspartyl protease, and compound C, an aspartyl protease transition-state analogue, have been reported to inhibit γ-secretase activity with equivalent potency for inhibition of Aβ₄₀ and Aβ₄₂ peptides (Shearman, M. S., et al., Biochem. 39: 8698-8704 (2000)). Time-dependant inhibition was observed when cells were incubated with compound C for various times up to twenty four hours (FIG. 4). In untreated cells (time zero minutes), significant NICD immunoreactivity was present in the nucleus and steadily decreased over time following exposure to 10 μM compound C. After three hours, NICD immunoreactivity in the nucleus declined further with no significant difference in levels of nuclear staining thereafter. Concurrently, cytoplasmic NICD staining steadily increased over time to reach maximal levels following twenty four hour exposure to compound C.

9. Simultaneous Detection of NICD Subcellular Localization and Quantification of Aβ Peptides

A key prerequisite for target-driven drug discovery is the ability to identify compounds that are active at the target site without significant off-target liability. As catalytic site-directed γ-secretase inhibition is associated with impaired Notch signalling (Lewis, H. D., et al., Biochem. 42:7580-7586 (2003)), an in vitro, cell-based assay enabling simultaneous detection of compound effects on Aβ₄₀, Aβ₄₂ peptide cleavage and Notch signalling would facilitate the identification of selective inhibitors. As such, the NICD nuclear translocation assay was modified to provide parallel measurements of Aβ₄₀ and Aβ₄₂ using cell-conditioned media.

For measurement of Aβ peptides, overnight cell incubation was required to generate sufficient levels of Aβ₄₀ and Aβ₄₂. However, this compromised the ability to perform single-cell image analysis due to increased cell proliferation and clustering associated with HEK293 cells. To overcome this, the serum concentration and cell seeding density were reduced to 0.4% (v/v) final and 10,000 cells/well, respectively. FIG. 5 shows the effect of catalytic-site directed γ-secretase inhibitor, compound D on Aβ peptide secretion and NICD nuclear translocation. The lack of separation between the IC₅₀ curves is indicative of the inability of Compound D to discriminate between Notch S3 and βAPP cleavages.

One skilled in the art would recognize that IC₅₀ values can be determined from a raw fluorescence intensity values by expressing fluorescence intensity data as the % control using the formula described above in Example 7. GraphPad Prism (GraphPad Software, Inc., San Diego, Calif.) can be used to plot the test compound concentration against the calculated % control. In that a sigmoidal dose response curve is typical for this type of analysis, a non-linear regression analysis is applied and Prism GraphPad is used to extrapolate the dose of compound which gives 50% inhibition, i.e., the IC₅₀ concentration. As long as a range of compound doses is measured and there is a graphical plateau at the beginning and end of the curve, similar IC₅₀ values will be obtained regardless of whether one uses the raw fluorescence intensity or the % control data.

10. NICD Nuclear Translocation is Maintained in the Presence of NSAID-Induced Aβ Modulation

The NSAID, compound E, has been reported to preferentially reduce Aβ₄₂ and this appears to be independent of cyclooxygenase (COX) inhibition (Weggen, S., et al., J. Biol. Chem 278: 31831-31837 (2003)). Without wishing to be bound by any theory, the mechanism of action for selective Aβ inhibition by NSAIDs is thought to be mediated by allosteric modulation of γ-secretase (Beher, D., et al., J. Biol. Chem. 279: 43419-43426 (2004)) and, thus, Notch S3 cleavage should remain unaffected. To test this hypothesis, cells were exposed to increasing concentrations of compound E for twenty four hours and Aβ₄₀, Aβ₄₂ and NICD nuclear translocation was quantified in the same samples (FIG. 6A). A dose dependant reduction in Aβ₄₂ was observed with no effect on nuclear translocation of NICD up to 100 μM. Aβ₄₀ levels also declined in response to increasing concentrations of compound C. Compound-induced cell cytotoxicity was observed at and above 30 μM (data not shown). Compound F, a COX inactive enantiomer of S-flurbiprofen, has also been shown to preferentially inhibit Aβ₄₂ (Eriksen, J. L., et al., J. Clin. Invest. 112: 440-449 (2003)). Treatment with compound F generated a similar selectivity profile (FIG. 6B) to that obtained with compound E (FIG. 6A) whereby Aβ₄₂ peptide levels were selectively reduced and NICD nuclear translocation was not inhibited at all tested concentrations up to 1000 μM. Aβ₄₀ peptide generation was inhibited at and above 30 μM compound F. In contrast, cells exposed to the γ-secretase inhibitor, compound G, demonstrated non-selective, dose-dependant inhibition of Aβ₄₀, Aβ₄₂ and NICD nuclear translocation (Table 1, below). This compound has been previously reported to exert significant Notch-related side effects upon chronic exposure in vivo (Wong, G. T., et al., J. Biol. Chem. 279: 12876-12882 (2004)). Comparable IC₅₀ values for inhibition of Aβ₄₀, Aβ₄₂ and NICD nuclear translocation were obtained for the γ-secretase inhibitor, compound G, (FIG. 6C) demonstrating a lack of discrimination for βAPP and Notch substrates. The NSAIDs, however, were inactive against Notch and inhibited Aβ₄₂ with reduced potency, consistent with an alternative mode of γ-secretase enzyme suppression.

11. Identification of Non-Selective Aβ Modulators

Having established and validated the assay, a screen was performed to identify novel compounds exhibiting selective inhibition of epsilon cleavage of βAPP without affecting Notch S3 cleavage. Two compounds, compounds H and I, were identified that demonstrated selective Aβ₄₀ and Aβ₄₂ reduction with no effect on Notch S3 cleavage (FIGS. 7A and 7B, respectively). Compound H inhibited Aβ₄₂ secretion and was less potent against Aβ₄₀ with no effect on NICD nuclear translocation up to 50 μM (FIG. 7A). In a similar fashion, compound I also selectively reduced Aβ₄₂ and was 10-fold less potent against Aβ₄₀ (FIG. 7B). Compound I had no effect on NICD nuclear translocation up to 5 μM although compound-induced cell cytotoxicity was noted at concentrations at and above 25 μM (data not shown). These non-selective modulators of Aβ may represent a new class of compounds with a mechanism of action separate and distinct from the Aβ₄₂-lowering NSAIDs.

TABLE 1

| | IC50 (nM) | | | | |
|---|---|---|---|---|---|
| Compound | Aβ40 | | Aβ42 | Notch | |
| Compound D | 4.7 | | 5.6 | 4.2 | |
| Compound E | >30 | μM | 450 | >100 | μM |
| Compound F | >1 | mM | 9240 | >1 | mM |

| Compound G | 0.033 | 0.034 | 0.034 | |
|---|---|---|---|---|
| Compound H | 204 | 13.4 | >100 | μM |
| Compound I | 806 | 56.97 | >5 | uM |

Selective inhibitors would be identified by calculating the IC₅₀ value for inhibition of Aβ₄₀, Aβ₄₂ and Notch and interpreting the values obtained to determine the selectivity profile of the compound. The IC₅₀ value is the concentration of compound that reduces a specified response to 50% of its former value (Oxford Dictionary of Biochemical and Molecular Biology, 2000). As used herein, “inactive compounds” are compounds which have not inhibited the response by 50% at the tested concentration range, but which might otherwise be deemed to have activity, albeit to a lesser degree. Those skilled in the art would generally consider an IC₅₀ value of less than 10 μM to be biologically significant for purposes of identify and selecting modulators of a given target in that compounds with values above 10 μM are perceived to be more likely to cause compound-induced cell cytotoxicity in a cell-based assay. Notwithstanding, IC₅₀ values up to 100 μM may be used to identify and select a sufficient number of candidates so as to give meaningful results. Those skilled in the art would understand that the IC₅₀ values obtained from in vitro assays are used to select compounds which are to be used in vivo and, as such, compounds exhibiting more potency based on their in vitro IC₅₀ values are perceived to have a higher probability of achieving suitable levels in vivo.

Based on the IC₅₀ values obtained herein (see, FIG. 8), the following parameters can be used for the identification of selective γ-secretase modulators:

(i) a compound having an IC₅₀ value less than 10 μM for inhibition of Aβ₄₂ and having no effect on Notch nuclear translocation at all tested concentrations, for example, an NSAID, would indicate a selective γ-secretase modulator;

(ii) a compound having an IC₅₀ less than 10 μM for both Aβ₄₀ and Aβ₄₂ and no effect on Notch nuclear translocation at all tested concentrations, for example, Compounds D and E, would indicate a selective γ-secretase modulator which does not discriminate between Aβ₄₀ and Aβ₄₂ inhibition;

(iii) a compound having substantially equal IC₅₀ values (within 10-fold difference) for Aβ₄₀, Aβ₄₂ and Notch, for example, Compounds C and G, would indicate a non-selective γ-secretase inhibitor;

(iv) a compound having substantially equal IC₅₀ values for Aβ₄₀ and Aβ₄₂ and an IC₅₀ value for Notch which was greater than 30 times the IC₅₀ for Aβ₄₀ and Aβ₄₂ (i.e. less potent) would indicate a Notch inhibitor;

(v) an IC₅₀ value for Notch inhibition only with no effect on Aβ₄₀ and Aβ₄₂ would indicate a selective Notch inhibitor; and

(vi) an IC₅₀ value for Aβ40 and Aβ42 with no effect on Notch activity at all tested concentrations would indicate a selective APP modulator.

What is claimed:

1. A method for identifying selective γ-secretase modulators using high content screening analysis of the translocation of a Notch intracellular domain (NICD) between the nucleus and cytoplasm in cells, which screening comprises:
(a) providing an array which contains multiple cells that express NICD and that are labeled with a detectable epitope tag;
(b) contacting the cells in said array with at least one test compound;
(c) imaging said cells in each location of the array with fluorescence optics;
(d) converting the optical information into digital data;
(e) analyzing said digital data to determine the degree of fluorescence in the nucleus and cytoplasm of said cells;
(f) calculating the test compound-induced changes to indicate one of the following:
i. a difference between the fluorescence from the reporter molecules in the cytoplasm from the fluorescence from the reporter molecules in the nucleus;
ii. a ratio of fluorescence from the reporter molecules in the cytoplasm to the fluorescence from the reporter molecules in the nucleus; and
wherein test compound-induced changes indicate an effect of the test compound on the translocation of NICD between the cytoplasm and the nucleus in the cells.

2. A method of claim 1 wherein said selective γ-secretase modulator is identified using a high content screening assay to simultaneously measure Aβ₄₀, Aβ₄₂ and Notch.

3. A method of claim 2 wherein a test compound that exhibits an IC₅₀ value for inhibition of Aβ₄₂ while exhibiting no effect on Notch nuclear translocation is identified as a selective γ-secretase modulator.

4. A method of claim 2 wherein a test compound that exhibits an IC₅₀ value solely for Notch inhibition and exhibiting no effect on Aβ₄₀ and β₄₂ is identified as a selective Notch inhibitor.

5. A method for identifying selective Notch modulators using high content screening analysis of the activation of Notch, which screening comprises:
a. providing an array which contains multiple cells that express full length Notch or cells capable of undergoing S2 ligand-dependent cleavage and that are labeled with a detectable fluorescent reporter molecule;
b. pre-incubating said cells with at least one test compound;
c. contacting said cells with at least one Notch binding ligand selected from the group consisting of Delta, Serrate and Jagged;
d. imaging said cells in each location of the array with fluorescence optics;
e. converting the optical information into digital data;
f. analyzing said digital data to determine the degree of fluorescence in the nucleus and cytoplasm of said cells;
g. calculating the test compound-induced changes to determine one of the following:
i. the difference between the fluorescence from the reporter molecules in the cytoplasm from the fluorescence from the reporter molecules in the nucleus;
ii. the ratio of fluorescence from the reporter molecules in the cytoplasm to the fluorescence from the reporter molecules in the nucleus; and
wherein test compound-induced changes identified in step (g) indicates i) an increase in the level of nuclear translocation of Notch in the cells pre-incubated with the test compounds as compared to control cells such that the test compound is a Notch receptor agonist, or ii) a decrease in the level of nuclear translocation of Notch in the cells pre-incubated with the test compounds as compared to control cells such that the test compound is a Notch receptor antagonist.

6. A method of treating Alzheimer's disease by administering to a patient in need of treatment thereof an effective amount of a selective γ-secretase modulator of claim 3.

7. A method of treating cancer and multiple sclerosis by administering to a patient in need of treatment thereof an effective amount of a selective Notch inhibitor of claim 4.
